(19) United States
(12) Patent Application Publication
(10) Pub. No.: US 2015/0164837 A1
(43) Pub. Date: Jun. 18, 2015

(54) USE OF AMPHOTERIC SURFACTANTS FOR THE PREVENTION AND TREATMENT OF PATHOGENIC VAGINAL BIOFILMS IN VAGINAL INFECTIONS

(71) Applicant: DR. AUGUST WOLFF GMBH & CO. KG ARZNEIMITTEL, BIELEFELD (DE)

(72) Inventors: Christoph Abels, Bielefeld (DE); Ulrich Knie, Bielefeld (DE); Michael Soeberdt, Bielefeld (DE)

(73) Assignee: DR. AUGUST WOLFF GMBH & CO. KG ARZNEIMITTEL, BIELEFELD (DE)

(21) Appl. No.: 14/385,230

(22) PCT Filed: Mar. 19, 2013
(86) PCT No.: PCT/EP2013/055706

§ 371 Date: Sep. 15, 2014

Related U.S. Application Data
(60) Provisional application No. 61/612,680, filed on Mar. 19, 2012.

(30) Foreign Application Priority Data
Mar. 19, 2012 (EP) 12160058.9

(51) Int. Cl. A61K 31/197; A61K 45/06

(52) U.S. Cl. CPC A61K 31/197; A61K 45/06

(57) ABSTRACT

The invention relates to amphotheric surfactants for use in preventing and/or treating vaginal infections, in particular for preventing and treating pathogenic vaginal biofilms in vaginal infections, as well as to pharmaceutical compositions containing the amphoteric surfactants as active ingredients.

### FIELD OF THE INVENTION

The invention relates to amphotheric surfactants for preventing and/or treating vaginal infections, in particular for preventing and/or treating pathogenic vaginal biofilms in vaginal infections. Moreover, the invention relates to pharmaceutical compositions containing at least one amphoteric surfactant for preventing and/or treating vaginal infections, and a pharmaceutically acceptable excipient.

### BACKGROUND OF THE INVENTION

Vaginal infections are one of the most common diseases diagnosed by gynaecologists. Vaginal infections may develop due to an imbalance of the healthy vaginal flora substantially composed of Lactobacilli. Lactobacilli are responsible for the acidic milieu of the vagina as well as for the production of substances inhibiting the growth of competing pathogenic agent. Due to the change of the normal milieu of the vagina caused by external or internal factors the pathogenic agents can proliferate causing the vaginal infection. Possible pathogenic agents may be bacilli, fungi or viruses.

Bacterial vaginosis (BV) is the most common microbiological disorder of the vaginal flora worldwide. Anaerobic bacteria, such as Gardnerella vaginalis overgrow the healthy Lactobacillus-dominant vaginal flora. This overgrowth leads to a decreased amount of Lactobacilli and an increased vaginal pH. Bacterial vaginosis is mainly found in women of reproductive age. The prevalence is about 5-15% in Caucasian, 45-55% in African and American blacks, and about 20-30% in Asian women. The incidence of bacterial vaginosis in pregnancy is about 10-20%, and often associated with adverse pregnancy outcomes like preterm birth or late miscarriage. Bacterial vaginosis is associated with an increased risk for STI (sexual transmitted infections) like HIV and genital herpes.

Only about 50% of women affected with bacterial vaginosis report symptoms. Women with symptomatic bacterial vaginosis are suffering from an unpleasant fishy smell and an increased watery, homogenous, grey vaginal discharge, but the vagina shows no signs of inflammation. For the diagnosis of a bacterial vaginosis, three of the following four Amsel criteria have to be fulfilled: homogeneous gray-white discharge, fishy smell, vaginal pH above 4.5 and clue cells on wet mount microscopy. Furthermore, the microscopic analysis of the vaginal fluid helps to diagnose bacterial vaginosis. For this purpose the vaginal fluid can be used stained and unstained. Gram staining is used to determine the Nugent score, which is referring to the number of Lactobacilli, small Gram-negative rods and curved Gram-variable rods found in the vaginal fluid. A Nugent score between 7 and 10 points indicates bacterial vaginosis. Microscopy of fresh unstained vaginal fluid shows, in case of existing bacterial vaginosis, a granular anaerobic flora, without leukocytes or the presence of parabasal cells (Donders, CME Review Article 2010, Vol. 65, No. 7).

Recently, the worldwide medical attention is more and more drawn to biofilm-related infections. Persisting infections and chronic diseases like obstructive lung disease, inflammatory bowel disease, bacterial endocarditis, periodontal disease, infections caused by indwelling medical devices (e.g. central venous catheters, urinary catheters, heart valves) and even gallstone formation are found to be induced or worsened by (developing and persisting) microbial biofilms (Donlan, Emerging Infectious Diseases, 2001, Vol. 7, No. 2; Ramage et al., Current Opinion in Infectious Diseases, 2010, 23: 560-565; Swidsinski et al., Obstetrics & Gynecology, Vol. 106, No. 5, Part 1, 2005).

Also in bacterial vaginosis (poly) microbial biofilms have been identified to potentially play a role in the recurrences of bacterial vaginosis. These biofilms are reported to consist mainly of Gardnerella vaginalis. One study showed that a polymicrobial biofilm, mainly composed of Gardnerella (60-95%), Atopobium (1-40%) and Lactobacilli (1-5%, in only 20% of the biofilm specimen), could be identified in 90% of women with bacterial vaginosis (Swidsinski et al., Obstetrics & Gynecology, Vol. 106, No. 5, Part 1, 2005).

Biofilms are defined in the art as surface-associated microbial communities, embedded in a matrix of extracellular polymeric substances (EPS). This matrix acts as protective barrier and enables the microbial cells to adhere to each other and to surfaces, such as metals, stones, plastics, aortic valves or catheters and human tissues. Biofilms can be found on nearly every kind of surfaces, all it takes to form biofilms are nutrition, moisture and microorganisms.

There are some remarkable differences between the planktonic (floating) and biofilm (sessile) form of bacteria. Bacterial species organized in a biofilm are more resistant to stress factors, like desiccation or UV-radiation, and in particular to antibiotic treatment as compared to the same species growing in planktonic form.

Biofilms are difficult to erase. There are several possible causes for this phenomenon. One explanation could be that most of the available antibiotics have only been tested for their killing activity against planktonic growing bacteria. Therefore the substances are very effective against bacteria in the planktonic, but mostly not effective against bacteria in the biofilm form. Other causes may be the great diversity of the biofilm bacteria, or the formation of “persister cells” that are metabolically quiescent. They are able to “turn off” the antibiotic targets like protein synthesis or DNA replication and are therefore resistant to antibiotics (Percival et al., Wound Rep Reg 2011, 19:1-9). These cells persist after the treatment and are able to recycle the existing EPS matrix as well as molecules and DNA fragments released by the dying cells, and thereby to build up a new biofilm (Costerton, The Biofilm Primer; 2007, Lewis et al., Annu Rev Microbiol 2010, 64:357:72).

Bacterial vaginosis usually is treated with antibiotics like metronidazole applied intravaginally or orally or clindamycin applied intravaginally. Even after correct treatment and an initially high cure rate of up to 94% (Brandt et al., EJOG 2008, 141:158-162), the recurrence rates are high. 30-50% of women experience a relapse within two to three months after the treatment with metronidazole or clindamycin (Vestraelen and Verhelst, Anti Infect. Ther, 2009, 7(9), 1109-1124). Thus in these cases, antibiotics can only slightly suppress the bacteria triggering bacterial vaginosis. For example, in a study 18 women with bacterial vaginosis were treated with oral metronidazole for 7 days. In a follow-up the presence and activity of bacterial biofilms were determined After the treatment for one week, patients showed no symptoms (discharge, malodour, clue cells). In a follow-up evaluation after 5 weeks the vaginal pH, the Nugent score, the biofilm density and the biofilm amenability were shown to increase over time. It was displayed that within the biofilm persistent Gardnerella vaginalis and Atopobium vaginae cells exist, which are relatively inaccessible to metronidazole. So in this study the vaginal biofilm was temporarily suppressed by metronidazole, but shortly after the treatment its activity was restored (Swidwinski et al., J. of Obstetrics & Gynecology 2008, 198:97.e1-97.e6). Gardner and Dukes were the first who showed, that the inoculation of vaginas of healthy women with material from vaginas of patients with Gardnerella vaginalis (back then Haemophilus vaginalis) infections with typical symptoms of bacterial vaginosis, and not the inoculation with isolated Gardnerella vaginalis bacteria, led to a clinical manifestation of bacterial vaginosis (back then H. vaginalis vaginitis) (Gardner and Dukes, Am J Obstet Gynecol 1955, 69(5):962-76). In more recent studies genomes of a commensal and a pathogenic isolate of Gardnerella vaginalis were compared. The pathogenic isolate showed a higher capacity to form biofilms. Thus the biofilm mode of life of Gardnerella vaginalis—in contrast to the planktonic growth—might be crucial for the pathogenesis and the recurrence of bacterial vaginosis (Harwich et al., BMC Genomics 2010, 11:375).

US2009/0181106A1 discloses a composition for treating biofilms using boric acid as agent with antimicrobial properties in combination with EDTA.

US 2006/0223765 discloses a method for inhibiting and/or treating infection in a vagina which employs as an active ingredient a saccharide-based non-ionic surfactant such as an alkyl-glycoside.

Catalone et al., Antimicrobial Agents and Chemotherapy, 2005, 49(4), 1509-1520, disclose the use of C31G (an equimolar mixture of alkyl dimethyl glycine and alkyl dimethyl amine oxide) as a vaginal microbiocide. Birnie et al., Antimicrobial Agents and Chemotherapy, 2000, 44(9), 2514-2517, and Journal of Pharmaceutical Sciences, 2001, 90 (9) disclose the evaluation of mixtures of alkyl betaines and alkyl dimethyl amine oxides of varying chain lengths with respect to their potential antimicrobial behaviour.

In view of the problems still existing in the prior art as described above, it is an object underlying the present invention to provide for an improved treatment/prevention of vaginal infections. In particular, it is an object to effectively treat and/or prevent bacterial vaginosis and, moreover, to erase biofilms (such as Gardnerella vaginalis biofilms) especially in women with relapsing bacterial vaginosis. It is particularly desired to avoid undesirable side effects and to significantly reduce the recurrence rate.

### SUMMARY OF THE INVENTION

The above mentioned problems have surprisingly been solved according to the present invention. Namely, it has surprisingly been found out that an amphoteric surfactant is able to effectively treat and/or prevent the formation of vaginal infections, in particular bacterial vaginosis being accompanied by the formation of (pathogenic) biofilms. It has especially been found out that persisting vaginal Gardnerella vaginalis (bacterial) biofilms can not only be effectively erased but also prevented. Therefore, amphoteric surfactants can be used to prevent and to treat (relapsing) bacterial vaginosis caused e.g. by Gardnerella vaginalis biofilms.

The effect of amphoteric surfactants on biofilms (such as Gardnerella vaginalis biofilms) is all the more remarkable, considering the fact that even a guideline-compatible therapy with an antibiotic like metronidazole or clindamycin does not remove the biofilm effectively, neither in vivo nor in a biofilm model. Likewise, a treatment of women with bacterial vaginosis with 400 mg moxifloxacin for five days just suppressed the adherent bacteria without eliminating them.

Consequently, the present invention relates to an amphoteric surfactant for use in the prevention and/or treatment of vaginal infections, and to a pharmaceutical composition comprising such an amphoteric surfactant as active ingredient for the prevention and/or treatment of the vaginal infections, and a pharmaceutically acceptable excipient.

### DETAILED DESCRIPTION OF THE INVENTION

The term surfactant is an abridgment of surface-active agent. Surfactants are amphipathic molecules, this means they consist of an oil- and a water-soluble group, i.e. a lipophilic and a hydrophilic part. Surfactants are classified by the hydrophilic part and its charge into non-ionic, cationic, anionic and amphoteric surfactants. The hydrophilic part of non-ionic surfactants is not charged. They do not comprise any dissociable functional groups, but one or more polar groups like ethers, ketones and alcohols. Often used examples of non-ionic surfactants are polyalkylene glycol ethers. Cationic surfactants are positively charged in their hydrophilic part. Most of them are quaternary ammonium compounds having halogenides as counterions, for example distearyldimethylammonium chloride. The hydrophilic part of anionic surfactants is negatively charged. They often bear carboxy, alkoxide, sulfonate or sulfate groups with alkali or alkaline atoms as counterions. An example of an anionic surfactant is sodiumlauryl sulfate.

The present invention relates to amphoteric surfactants and to pharmaceutical compositions containing the same as active ingredients. In general, depending on the pH value the hydrophilic part of an amphoteric surfactant contains at least one group that is or can be positively charged as well as at least one group that is or can be negatively charged. The groups that are or can be positively charged are for example amines or ammonium compounds. The groups that are or can be negatively charged are for example carboxy, carboxylate, sulfonate or sulfate groups.

An advantage of amphoteric surfactants is their harmlessness. Their safety is proved by the use in many cosmetics, like shampoos or shower gels. Further, in contrast to non-ionic and anionic surfactants amphoteric surfactants are well tolerated by the skin due to their mildness, and the absence of irritant effects.

Amphoteric surfactants which can be used according to the present invention in the prevention and/or the treatment of vaginal infections comprise in addition to a lipophilic part, preferably a long chain (such as C₆-C₂₄) alkyl or acyl group, at least one amine or ammonium function and at least one group selected from —COOH and —SO₃H to be capable of forming inner salts. Preferable amphoteric surfactants are amphoacetates, amphodiacetates, amphopropionates, amphodipropionates, sulfobetaines, and hydroxysultaines (according to INCI nomenclature: European Commission database with information on cosmetic substances and ingredients (CosIng)).

In a preferred embodiment the amphoteric surfactant of the present invention is an amphoacetate, amphodiacetate, amphopropionate, an amphodipropionate, a hydroxysultaine, or a mixture thereof. These amphoteric surfactants have been shown to be particularly well tolerated and do not cause skin irritation, a property being highly important for treating vaginal disorders since the vaginal mucous membrane is highly sensitive.

In a further preferred embodiment the amphoteric surfactant of the present invention is an alkylamphoacetate, alkylamphodiacetate, alkylamphopropionate, alkylamphodipropionate and/or an alkylamidopropyl hydroxysultaine, preferably a C₆-C₂₄ alkylamphoacetate, C₆-C₂₄ alkylamphodiacetate, C₆-C₂₄ alkylamphopropionate, C₆-C₂₄ alkylamphodipropionate and/or a C₆-C₂₄ alkylamidopropyl hydroxysultaine, more preferably C₈-C₁₈ alkylamphoacetate, C₈-C₁₈ alkylamphodiacetate, C₈-C₁₈ alkylamphopropionate, C₈-C₁₈ alkylamphodipropionate and/or a C₈-C₁₈ alkylamidopropyl hydroxysultaine. Even more preferably the amphoteric surfactant of the present invention is selected from the group consisting of cocoamphoacetate, lauroamphoacetate, caproamphoacetate, caprylamphoacetate, stearoamphoacetate, isostearoamphoacetate, myristoamphoacetate, cocoamphodiacetate, lauroamphodiacetate, caproamphodiacetate, caprylamphodiacetate, stearoamphodiacetate, isostearoamphodiacetate, myristoamphodiacetate, cocoamphopropionate, lauroamphopropionate, caproamphopropionate, caprylamphopropionate, stearoamphopropionate, isostearoamphopropionate, myristoamphopropionate, cocoamphodipropionate, lauroamphodipropionate, caproamphodipropionate, caprylamphodipropionate, stearoamphodipropionate, isostearoamphodipropionate, myristoamphodipropionate, cocoamidopropyl hydroxysultaine, lauramidopropyl hydroxysultaine, capramidopropyl hydroxysultaine, caprylamidopropyl hydroxysultaine, stearamidopropyl hydroxysultaine, isostearamidopropyl hydroxysultaine, and myristamidopropyl hydroxysultaine. According to the invention any mixture of the above mentioned amphoteric surfactants can be employed as well.

In a preferred embodiment the amphoteric surfactant of the present invention is a C₆-C₂₄ alkylamphoacetate, preferably selected from cocoamphoacetate, lauroamphoacetate, caproamphoacetate, caprylamphoacetate, stearoamphoacetate, isostearoamphoacetate, and myristoamphoacetate, or any mixtures thereof.

In a further preferred embodiment the amphoteric surfactant of the present invention is a C₆-C₂₄ alkylamphodiacetate, preferably selected from cocoamphodiacetate, lauroamphodiacetate, caproamphodiacetate, caprylamphodiacetate, stearoamphodiacetate, isostearoamphodiacetate, and myristoamphodiacetate, or any mixtures thereof.

In a further preferred embodiment the amphoteric surfactant of the present invention is a C₆-C₂₄ alkylamphopropionate, preferably selected from cocoamphopropionate, lauroamphopropionate, caproamphopropionate, caprylamphopropionate, stearoamphopropionate, isostearoamphopropionate, and myristoamphopropionate, or any mixtures thereof.

In a further preferred embodiment the amphoteric surfactant of the present invention is a C₆-C₂₄ alkylamidopropyl hydroxysultaine, preferably selected from cocoamidopropyl hydroxysultaine, lauramidopropyl hydroxysultaine, capramidopropyl hydroxysultaine, caprylamidopropyl hydroxysultaine, stearamidopropyl hydroxysultaine, isostearamidopropyl hydroxysultaine, and myristamidopropyl hydroxysultaine, or any mixtures thereof.

Most preferably the amphoteric surfactant of the present invention is a cocoamphoacetate or a lauroamphoacetate, preferably sodium cocoamphoacetate or sodium lauroamphoacetate, a cocoamphopropionate, preferably sodium cocoamphopropionate, a cocoamphodiacetate, preferably disodium cocoamphodiacetate or cocoamidopropyl hydroxysultaine.

Particularly, each of sodium cocoamphoacetate, sodium lauroamphoacetate, sodium cocoamphopropionate, disodium cocoamphodiacetate and cocoamidopropyl hydroxysultaine both (a) show excellent results in preventing and/or treating vaginal infections (in particular those with vaginal biofilms) as well as (b) provide superior characteristics in terms of skin toleration (non-irritating behaviour).

According to the present invention the above-mentioned amphoteric surfactants are used as active compounds for the prevention and/or the treatment of vaginal infections.

Vaginal infections as defined according to the present invention comprise any infectious states or disorders of the vagina with undesirable characteristics. The vaginal infections may be caused by fungi (such as Candida albicans and Candida spec), or bacteria (such as Gardnerella vaginalis, Atopobium vaginae, Mobiluncus spp., Prevotella spp., and Mykoplasma hominis). Vaginal infections as defined according to the present invention are preferably accompanied by (pathogenic) vaginal biofilms. “Biofilms” are defined herein as surface-associated microbial communities, embedded in a matrix of extracellular polymeric substances (EPS). “Accompanied by” means herein the appearance of vaginal biofilms in association with vaginal infections. It is important to note that vaginal biofilms are not inherent to vaginal infections in a general manner. Quite in contrast, vaginal biofilms are found in association with specific vaginal infections only. Vaginal biofilms have been observed with vulvovaginal candidiasis or bacterial vaginosis. These diseases, if showing the presence of vaginal biofilms, are preferred conditions to be treated and/or prevented according to the present invention. A particularly preferred condition according to the invention is bacterial vaginosis accompanied by a vaginal biofilm that is caused by Gardnerella vaginalis.

It has been surprisingly found out according to the present invention that the vaginal biofilms as mentioned above are effectively erased upon application of the amphoteric surfactants. Moreover, the biofilms are not only effectively erased but the rate of their formation is significantly reduced. Thus, the amphoteric surfactants are effective not only in the treatment but also in prevention of vaginal infections, in particular when accompanied by vaginal biofilms. Without being bound by theory, the inventors believe, that adding amphotheric surfactants to the biofilm destroys its EPS structure and therefore releasing biofilm bacteria out of their protective environment. The exposed pathogenic germs are now amenable for bactericidal substances (pharmaceutically active substances, such as antibiotic and antiseptic substances or even amphoteric surfactants) and can therefore be killed more easily. In particular, the quiescent cells, also referred to as viable, but not culturable cells, are forced to take up metabolic activity, thus becoming also amenable to conventional antibiotic or antimycotic treatment. In addition, surprisingly the surfactant will not only interact and destroy the EPS, but will also interact directly with substances of the cell membrane, further affecting the viability of the pathogenic agents.

The vaginal infections to be treated and/or prevented according to the present invention are preferably characterized by a loss of lactobacilli. In a healthy vaginal milieu there are enough lactobacilli to ensure the acidic milieu and to inhibit the growth of unpleasant other pathogenic germs. In case the balance of the vaginal milieu is changed due to an inner or outer influence in disadvantage to lactobacilli, then an uncontrolled growth of unpleasant pathogenic germs, for example Gardnerella vaginalis, Atopobium vaginae or Candida spec. can occur eventually leading to a vaginal infection.

In a preferred embodiment of the invention the amphoteric surfactant is applied in an amount of 0.01 to 500 mg per dose. Preferably, the amphoteric surfactant is applied in an amount of 0.1 to 250 mg, especially of 1 to 100 mg per dose. If the amount is below the above values, the treatment and/or prevention is less effective. On the other hand, if the amount is above the values mentioned, skin irritation may be observed.

The amphoteric surfactant according to the invention may be topically applied to the vagina and/or the vulva. It is particularly preferred to bring the amphoteric surfactant into direct contact with the pathogenic biofilm, if present. Moreover, the amphoteric surfactant in accordance with the invention may be applied in form of an ointment, a cream, a gel, a tablet, a capsule, an ovule, a suppository, a solution, a suspension, a foam, a film or liposomal composition. Particularly preferable application forms are ointments, gels, creams and suppositories.

It is also possible according to the invention to apply the amphoteric surfactant by a vaginal ring, tampon, sponge, pillow, puff, or osmotic pump system. For these purposes the before-mentioned articles may be impregnated with the amphoteric surfactant or may be dipped in a cream or an ointment containing the amphoteric surfactant.

The present invention also relates to a pharmaceutical composition containing an amphoteric surfactant as defined herein as an active ingredient for the treatment and/or prevention of vaginal infections, and a pharmaceutically acceptable excipient. With respect to the amphoteric surfactant and the vaginal infections the statements given above apply for the pharmaceutical composition as well. It is important to note that the amphoteric surfactant according to the present invention has surprisingly been found as an active ingredient for the treatment and/or prevention of the vaginal infections.

In a preferred embodiment the pharmaceutical composition contains 0.1 to 15 wt. %, preferably 0.25 to 10 wt. %, more preferably 0.5 to 7.5 wt. % of an amphoteric surfactant or a mixture thereof, based on the total weight of the pharmaceutical composition.

The pharmaceutical composition of the present invention is preferably applied such that 0.01 to 500 mg of amphoteric surfactant is used per application. In a more preferred embodiment 0.1 to 250 mg, even more preferably 1 to 100 mg of amphoteric surfactant are used per application.

As mentioned, the pharmaceutical composition of the invention further comprises at least one pharmaceutically acceptable excipient that facilitates/enables the drug administration at a convenient site. Pharmaceutically acceptable excipients suitable according to the invention are those known to the skilled person, in particular those for the modes of application/administration as described already above for the amphoteric surfactant per se. Preferably the one or more pharmaceutical acceptable excipients are chosen from solvents, gelling agents, buffers, non amphoteric surfactants (e.g. anionic, cationic, and/or non-ionic surfactants), detergents, oils, alcohols, emulsifiers, solubilizers, humectants, fillers, carriers and bioadhesives.

Suitable excipients may be inorganic or organic substances for topical and vaginal administration, preferably for vaginal and/or vulvar administration. Since this area is very sensitive the excipient has to be very soft to the skin. Examples of particularly preferred excipients are water, plant oils, benzyl alcohols, polyethylene alcohols/glycols, gelatine, soya, carbohydrates (such as lactose or starch), lecithin, glycerol triacetate and other fatty acid glycerides, talc and cellulose. Examples of gelling agents as suitable excipients are natural gelling agents, such as pectin, agarose, gelatine and casein, or modified natural gelling agents, such as methyl cellulose, hydroxymethyl cellulose, hydroxymethylpropyl cellulose and carboxymethyl cellulose or full synthetic gelling agents, such as polyvinylalcohols, poly(meth)acrylacids, polyacrylamide, polyvinylpyrrolidone and polyethylene glycole.

Any further suitable pharmaceutical excipients known to the skilled person may preferably be added such as perfumes, preservatives, colorants, etc.

In a preferred embodiment the pharmaceutical composition further contains an acid to adjust the pH of the pharmaceutical composition in the range of 3 to 6 and more preferably in the range of 4 to 5. Preferably the acid used in the pharmaceutical composition according to the present invention is an organic acid, for example lactic or citric acid. Lactic acid is especially preferred. Acids may be present in the present pharmaceutical composition from 0 to 5 wt. %, more preferably from 0.01 to 0.5 wt. %, based on the total weight of the pharmaceutical composition. Preferably also buffers can be added to ensure the pH value remaining in the above-mentioned range. Such a buffer may be acetic acid/acetate buffer.

More preferably, the pharmaceutical composition may be applied from once a day to twice a week. For treating the acute vaginal infections, for example caused by Gardnerella vaginalis biofilms, the pharmaceutical composition is preferably applied once a day for one or two weeks. For preventing the formation of new biofilms the treatment with the pharmaceutical composition can be preferably applied twice a week, for example over a period of several months.

Preferably, the pharmaceutical composition/the amphoteric surfactant of the present invention can be co-administered with other pharmaceutically active substances such as antibiotics or antiseptic agents. Preferred antibiotics are metronidazole or clindamycin. The amphoteric surfactant may preferably be administered simultaneous with, before and/or after the treatment with the antibiotics, in order to further improve the treatment and/or prevention of vaginal infections. In a preferred embodiment the amphoteric surfactant and the further pharmaceutically active substance are administered one after the other. The pharmaceutically active substance (such as an antibiotic or an antiseptic) is given first, after ending the antibiotic or antiseptic therapy the amphoteric surfactant is administered, or vice versa. Furthermore, a synergistic effect is assumed by administering both, a further pharmaceutically active substance and an amphoteric surfactant. First the pharmaceutically active substance (e.g. an antibiotic or an antiseptic) is killing the amenable bacteria, then the amphoteric surfactant is destroying the EPS structure of the biofilm. Adjacent the administration of another pharmaceutically active substance will kill the remaining bacteria, which are now more amenable, without the protecting EPS. When administered vice versa the amphoteric surfactant will destroy the EPS structure of the biofilm first and then the pharmaceutically active substance will kill the amenable bacterial.

Therefore the administration of the amphoteric surfactant and the further pharmaceutically active substance can also be splitted into different therapy regimen:
a) the further pharmaceutically active substance (such as an antibiotic, or an antiseptic) is administered first for an appropriate time, depending on the pharmaceutically active substance as indicated in the respective patient information leaflets, for example, upon completion therapy with the further pharmaceutically active substance, the application of the amphoteric surfactant follows. The amphoteric surfactant can be applied once daily for one or two weeks, or twice a week for several months. After completing the ampotheric surfactant therapy a second therapy with a pharmaceutically active substance (such as an antibiotic, or an antiseptic) follows for an appropriate time as indicated in the respective patient information leaflets, for example.
b) the amphoteric surfactant is administered first, for one or two weeks once a daily, or for several months, twice a week. Afterwards the further pharmaceutically substance (such as an antibiotic or an antiseptic) can be administered for an appropriate time depending on the pharmaceutically active substance as indicated in the respective patient information leaflets, for example.

In addition the pharmaceutical composition of the present invention can be used in patients whose infections (such as Gardnerella vaginalis biofilm infections) have failed to respond to other antibiotics or antimicrobials.

### BRIEF DESCRIPTION OF THE DRAWINGS

FIG. 1 is a diagram that shows the inhibition of Gardnerella vaginalis biofilm formation and viability by sodium cocoamphoacetate.

FIG. 2 is a diagram displaying that sodium cocoamphoacetate inhibits the formation and viability of Gardnerella vaginalis stationary biofilms.

FIG. 3 is a diagram that shows the inhibitory effect of several amphoteric surfactants added to a mature Gardnerella vaginalis biofilm

### EXAMPLES

### Example 1

### Inhibition of G. vaginalis Biofilm Formation and Viability by Sodium Cocoamphoacetate in Forming as Well as Well Developed but Still Growing G. vaginalis Biofilms

### A) Experimental Methods

The inhibitory effect of amphoteric surfactants on the formation of Gardnerella vaginalis biofilms were tested in biofilms, grown in 96 well tissue culture test plates. Before starting the biofilm experiments, MIC (minimal inhibitory concentration) values for sodium cocoamphoacetate for planktonic growing Gardnerella vaginalis cultures were determined MIC values for sodium cocoamphoacetate against Gardnerella vaginalis had not been reported so far.

Gardnerella vaginalis (strain ATCC 14018) were grown on Columbia agar plates supplemented with 5% sheep blood, liquid cultures were grown in brain heart infusion broth supplemented with 2% (w/v) gelatine, 0.5% yeast extract, 0.1% starch and 1% D-(+)-glucose at 37° C. and with the addition of 5% CO₂.

For a preculture, bacteria were inoculated from plate or glycerol stock and grew overnight. This preculture was used for starting a new culture. Here the cultivation was performed in microtiter plates in a final volume of 2004 For testing substances in a forming biofilm (t0), substances were added at this time point. After 20 hours of cultivation, measurements determining the mass of biofilm cells and the biofilm viability were started.

For testing substances in a well developed and further growing biofilm (t20m), medium was gently removed after 20 hours of cultivation and compounds were added in fresh medium for enabling further growth. Incubation was performed for another 20 hours.

At 20 hours (t0) respectively 40 hours of cultivation (t20m) measurements were started, concerning the biofilm formation and viability.

Crystal violet (CV) staining was used to determine the mass of biofilm cells. For this purpose biofilms grown in 96 well tissue test plates were washed with buffer (PBS, pH of 7.4), dried and stained under shaking with 2% crystal violet in ethanol. After washing with buffer, drying and over night extraction with ethanol, the absorbance (OD₆₂₀) was measured in a plate reader and the percentage of biomass inhibition was calculated.

The viability was determined using the Live/Dead BacLight bacterial viability staining. This method utilizes mixtures of SYTO 9 green-fluorescent nucleic acid stain and the red-fluorescent nucleic acid stain, propidium iodide. These stains differ both in their spectral characteristics and in their ability to penetrate viable bacterial cells. When used alone, the SYTO 9 stain generally labels all bacteria in a population, those with intact membranes and those with damaged membranes. In contrast, propidium iodide penetrates only bacteria with damaged membranes, causing a reduction in the SYTO 9 stain fluorescence when both dyes are present. Biofilms grown in 96 Well Optical plates were carefully washed with 0.85% NaCl and air-dried. Afterwards 100 μl of the staining solution (containing 6 μM SYTO 9 stain and 30 μM propidium iodide in 0.85% NaCl) were added. After 15 min of incubation in the dark fluorescence was measured in a microtiter plate reader equipped with detectors and filter sets for monitoring red (630 nm) and green (530 nm) fluorescence. Afterwards the percentage of the reduction of viability was calculated.

### B) Inhibition of Gardnerella vaginalis Biofilm Formation and Viability by an Amphoteric Surfactant

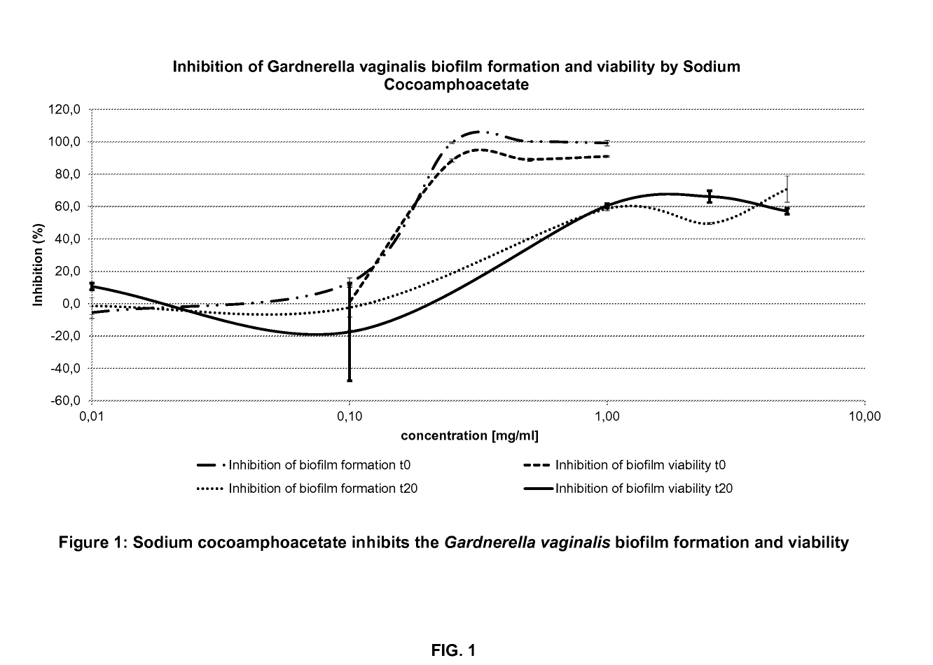

FIG. 1 displays the inhibitory effect of sodium cocoamphoacetate on Gardnerella vaginalis biofilm formation and viability, in different stages of biofilm development.

When sodium cocoamphoacetate was added at the beginning of biofilm cultivation (t0), the biofilm formation was completely inhibited with concentrations of 0.25 mg/ml (that means mg cocoamphoacetate/ml medium) and higher.

Adding the amphoteric surfactant to a well developed and still growing Gardnerella vaginalis biofilm (t20m) the concentration needed to inhibit the biofilm formation was higher. With concentrations between 1 mg/ml to 5 mg/ml a 36% to 71% reduction in biofilm formation was achieved.

With the addition of sodium cocoamphoacetate (0.25 mg/ml to 1 mg/ml) at the beginning of the biofilm cultivation (t0), the viability of the bacterial cells was decreased by 90%. When added to a well developed and growing biofilm, a 38% to 67% reduced viability of bacterial cells was shown with concentrations between 1 mg/ml to 5 mg/ml.

Surprisingly these findings indicate that the addition of sodium cococamphoacetate to a developing Gardnerella vaginalis biofilm prevents the formation of a new biofilm by massive reduction of the cell mass and viability. Unexpectedly sodium cocoamphoacetate also inhibits the biofilm formation and the cell viability in well developed and still growing biofilms, thus pharmaceutical compositions containing this amphoteric surfactant must be considered as further/additional possible treatment against Gardnerella vaginalis infections, especially when an existing Gardnerella vaginalis biofilm is involved/can be detected.

### Comparative Example

As reference a study of Swidsinski, et al., 2008 (Am J Obstet Gynecol 2008; 198:97.e1-97.e6) is mentioned. In this study 18 women with confirmed bacterial vaginosis (fulfilling all 4 Amsel criteria, median Nugent Score=9), were treated with 500 mg metronidazole orally, twice daily for 7 days. Follow-up visits, were performed during the treatment at day 3 or after the treatment at day 7, 14, 21, 28, and 35, during these follow-up visits vaginal biopsies were taken. The authors visualised the vaginal biofilms via FISH (fluorescent in situ hybridization) technique.

Even though, after a 7 day treatment period with oral metronidazole, the patients seemed to be clinically cured (they remained free of vaginal discharge, malodour, clue cells and the Nugent Score was below 7), the biopsies showed an accumulation of Gardnerella vaginalis and Atopobium vaginae in an adherent biofilm that grew and became more prominent over time.

This comparative example showed in an impressive way that the standard therapy for bacterial vaginosis failed to erase the existing vaginal biofilm.

### Example 2

### Inhibition of Stationary Gardnerella vaginalis Biofilms by Sodium Cocoamphoacetate

### A) Experimental Methods

To determine how different concentrations of sodium cocoamphoacetate damage well formed biofilms in the stationary phase, compounds were added in PBS buffer to 20 hours old biofilms and analyzed after further 20 hours of incubation by determining their biomass and viability.

### B) Inhibition of a Gardnerella vaginalis Biofilm in the Stationary Phase

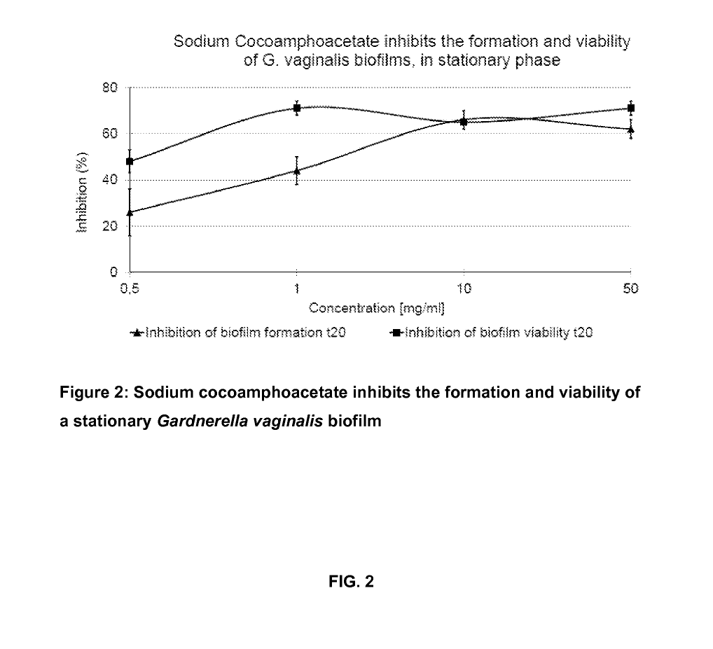

FIG. 2 illustrates that sodium cocoamphoacetate influences/inhibits both the formation and the viability of Gardnerella vaginalis biofilms in the stationary phase.

Bacterial biofilms in stationary phase are known to be highly resistant to antibiotic treatments.

Sodium cocoamphoacetate was tested for its ability to inhibit formation and viability of Gardnerella vaginalis biofilms in the stationary phase as well. Surprisingly, the addition of sodium cocoamphoacetate in concentrations from 1 mg/ml to 50 mg/ml (that means mg cocoamphoacetate/ml medium) decreased the biofilm viability by about 70%, and furthermore a reduction in biofilm formation by 44-65% was achieved.

These findings indicate that the addition of sodium cocoamphoacetate to Gardnerella vaginalis biofilms in stationary phase is surprisingly an outstanding method for reducing the cell viability and the cell mass of G. vaginalis biofilms in stationary phase.

### Example 3

### Inhibition of Gardnerella vaginalis Biofilm by Further Amphoteric Surfactants

The tested substances namely cocoamidopropyl hydroxysultaine, disodium cocamphodiacetate, sodium cocoamphopropionate and sodium lauroamphoacetate were dissolved in fresh media and added to a mature G. vaginalis biofilm (t20m, see Example 1). After another 20 hrs of incubation, the biofilm viability was determined via Live/Dead staining, as described above.

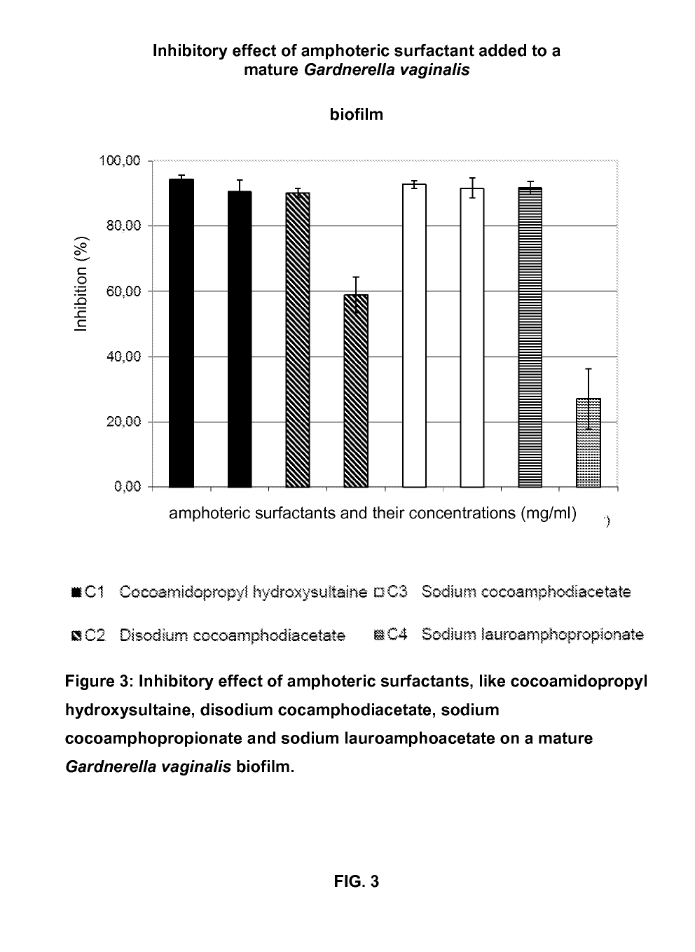

The used compounds were added in several concentrations, whereas only two different concentrations per compound are shown in FIG. 3.

It was surprisingly found that the added amphoteric surfactants also inhibited the biofilm formation and the cell viability in well developed and still growing biofilms. In particular cocoamidopropyl hydroxysultaine and sodium cocoamphopropionate are good candidates, because even low concentrations (0.14 mg/ml and 0.16 mg/ml respectively) can inhibit the biofilm viability up to 90%.

Summarizing all given Examples the surprising findings according to the present invention show that amphoteric surfactants like sodium cocoamphoacetate, cocoamidopropyl hydroxysultaine, disodium cocamphodiacetate, sodium cocoamphopropionate and sodium lauroamphoacetate are able to reduce the cell mass and to inhibit the viability of well developed and still growing Gardnerella vaginalis biofilms. Further studies (cf. Comparative Example) illustrated that 500 mg metronidazole administered orally twice daily did not erase the Gardnerella vaginalis biofilm completely. Therefore, amphoteric surfactants such as sodium cocoamphoacetate, cocoamidopropyl hydroxysultaine, disodium cocamphodiacetate, sodium cocoamphopropionate and sodium lauroamphoacetate are potent substances for the treatment of relapsing vaginal infections due to vaginal biofilms.

Examples 1 to 3 clearly show the unexpected inhibition of growing and stationary Gardnerella biofilm after the addition of an amphoteric surfactant according to the present invention.

### Examples for Compositions/Formulations According to the Present Invention

### Ointment

| | |
|---|---|
| Sodium cocoamphoacetate (30%) | 5.0 g |
| Lactic acid (90%) | 0.5 g |
| Macrogol 300 | 45.0 g |
| Macrogol 1500 | 49.5 g |

Suppository/Ovula:

| | |
|---|---|
| Sodium cocoamphoacetate (30%) | 0.1 g |
| Lactic acid (90%) | 0.01 g |
| Macrogol 1500 | 2.0 g |
| Macrogol 1000 | 0.7 g |

Cream:

| | |
|---|---|
| Sodium cocoamphoacetate (30%) | 5.0 g |
| Lactic acid (90%) | 0.5 g |
| Cetostearyl alcohol | 7.0 g |
| Macrogol 6 cetostearyl ether | 1.5 g |
| Macrogol 25 cetostearyl ether | 1.5 g |
| Liquid paraffin | 12.0 g |
| Parabenes | 0.2 g |
| Propylene glycol | 8.0 g |
| Water | 64.3 g |

Gel:

| | |
|---|---|
| Sodium cocoamphoacetate (30%) | 5.0 g |
| Lactic acid (90%) | 0.5 g |
| Phenoxyethanol | 1.0 g |
| Propylene glycol | 3.0 g |
| Methylhydroxypropylcellulose | 2.0 g |
| Water | 88.5 g |

1. An amphoteric surfactant for use in the prevention and/or treatment of vaginal infections.

2. The amphoteric surfactant according to claim 1, which is an amphoacetate, an amphodiacetate, an amphopropionate, an amphodipropionate, a hydroxysultaine, or any mixtures thereof.

3. The amphoteric surfactant according to claim 2, wherein the surfactant is Chosen from the group consisting of a C₆-C₂₄ alkylamphoacetate, a C₆-C₂₄ alkylamphopropionate, a C₆-C₂₄ alkylamidopropyl hydroxysultaine, and a C₆-C₂₄ amphodiacetate.

4. The amphoteric surfactant according to claim 2, wherein the vaginal infection is characterized by the presence of a vaginal biofilm.

5. The amphoteric surfactant according to claim 4, wherein the vaginal infection is vulvovaginal candidiasis or bacterial vaginosis.

6. The amphoteric surfactant according to claim 4, wherein the vaginal biofilm is caused by Candida albicans and Candida spec. and/or Gardnerella vaginalis, Atopobium vaginae, Mobiluncus spp., Prevotella spp., and Mykoplasma hominis.

7. The amphoteric surfactant according to claim 2, wherein the amphoteric surfactant is applied in at amount of 0.01 to 500 mg per dose.

8. The amphoteric surfactant according to claim 7, wherein the amphoteric surfactant is applied from once a day to twice a week.

9. The amphoteric surfactant according to claim 8, wherein the amphoteric surfactant is applied in form chosen from the group consisting of an ointment, a cream, a gel, a tablet, a capsule, an ovule, a suppository, a. solution, a suspension, a foam, a film or liposomal composition, and being contained within a vaginal ring, tampon, suppository, sponge, pillow, puff, or osmotic pump system.

10. The amphoteric surfactant according to claim 9, wherein the amphoteric surfactant is topically applied to the vagina and/or the vulva.

11. A pharmaceutical composition comprising:
(a) an amphoteric surfactant according to claim 2 as active ingredient for the prevention and/or treatment of vaginal infections, and
(b) a pharmaceutically acceptable excipient.

12. The pharmaceutical composition according to claim 11, which comprises 0.1 to 15 wt. % of the amphoteric surfactant, based on the total weight of the pharmaceutical composition.

13. The pharmaceutical composition according to claim 12, wherein the pharmaceutically acceptable excipient is selected from the group consisting of solvents, gelling agents, buffers, non amphoteric surfactants, detergents, oils, alcohols, emulsifiers, solubilizers, humectants, fillers, carriers and bioadhesives.

14. The pharmaceutical composition according to claim 13 further comprising an additional therapeutically active substance.

15. The pharmaceutical composition according to claim 14, wherein the additional therapeutically active substance is an antibiotic.

16. The amphoteric surfactant according to claim 3, wherein the surfactant is chosen from the group consisting of cocoamphoacetate, lauroamphoacetate, sodium cocoamphoacetate, sodium lauroamphoacetate, a cocoamphopropionate, sodium cocoamphopropionate, cocoamidopropyl hydroxysultaine, a cocoamphodiacetate, and a sodium cocoamphodiacetate.

17. The amphoteric surfactant according to claim 7, wherein the amphoteric surfactant is applied in an amount of 0.1 to 250 mg per dose.

18. The amphoteric surfactant according to claim 17, wherein the amphoteric surfactant is applied in an amount of 1 to 100 mg per dose.

19. The pharmaceutical composition according to claim 15, wherein the additional therapeutically active substance is selected from the group consisting of metronidazole, clindamycin, moxifloxacin, and an antiseptic.

20. The pharmaceutical composition according to claim 19, wherein the antiseptic is chosen from the group consisting of povidone-iodine, hexitidine, and octinidine.
